(19) United States
(12) Patent Application Publication
(10) Pub. No.: US 2021/0189288 A1
(43) Pub. Date: Jun. 24, 2021

(54) ANTIOXIDANT FORMULATIONS

(71) Applicant: KEMIN INDUSTRIES, INC., Des Moines, IA (US)

(72) Inventors: Sara Cutler, San Angelo, TX (US); Ewa Szajna-Fuller, Ames, IA (US); Isabella Rotberg, Boynton Beach, FL (US); Carrie Wray, Des Moines, IA (US); My Truong, Pleasant Hill, IA (US); Mitchell Poss, Johnston, IA (US)

(21) Appl. No.: 17/193,869

(22) Filed: Mar. 5, 2021

Related U.S. Application Data
(63) Continuation of application No. 13/608,812, filed on Sep. 10, 2012.
(60) Provisional application No. 61/532,859, filed on Sep. 9, 2011.

(51) Int. Cl. C11B 5/00; A61K 36/82; A61K 36/537; A61K 36/534; A61K 36/53; A23K 20/111; A23K 20/105; A23K 20/174; A23K 50/40; A23K 20/10; A23K 20/158

(52) U.S. Cl. CPC C11B 5/0092; A61K 36/82; A61K 36/537; A61K 36/534; A61K 36/53; A23K 20/158; A23K 20/105; A23K 20/174; A23K 50/40; A23K 20/10; A23K 20/111

(57) ABSTRACT

Antioxidant formulations containing new active molecules with tocopherols are disclosed. The best performing formulas contain extracts of green tea that are oil soluble, extracts of rosemary, extracts of spearmint and tocotrienols. Interestingly, the amount of tocopherols in formulas could be reduced by 50% in this diet when the other actives were increased accordingly.

### CROSS-REFERENCE TO RELATED APPLICATIONS

This application claims priority to U.S. patent application Ser. No. 13/608,812, filed Sep. 10, 2012, entitled “ANTIOXIDANT FORMULATIONS,” which claims the benefit of priority to U.S. Provisional Patent Application No. 61/532,859, filed Sep. 9, 2011, both of which are incorporated herein by reference in their entireties.

### BACKGROUND OF THE INVENTION

The present invention relates generally to antioxidant formulations containing extracts of tea and extracts of spearmint and, more specifically, to antioxidant formulations for pet food containing lipid-soluble extracts of tea and water-soluble extracts of a Lamiaceae spp. plant such as spearmint containing, inter alia, rosmarinic acid.

Antioxidants are applied at several stages of the pet food kibble manufacturing process before, during, and after extrusion. One common antioxidant comprises mixed tocopherols and/or tocotrienols. In one particular example, Naturox® Plus Dry (Kemin Industries, Inc., Des Moines, Iowa) is a dry antioxidant (DA) mixture which is added to the kibble dry recipe before it is extruded, while Naturox® Premium Liquid (Kemin Industries, Inc., Des Moines, Iowa) is a liquid antioxidant (LA) formulation in oil added to the enrobing fat on the kibble's surface. Since LA is applied closest to the air/kibble interface, it is crucial to the oxidative stability of the kibble. The LA formulation may also be applied to the meat meal during its production in the rendering process or directly after the rendering when the meal is isolated from the offal, to control oxidation of protein and fat prior to be used in the dry meal.

Green and black teas, as well as other varieties of tea, are well known to have water soluble antioxidants which perform well in a hydrophilic food matrix. Previous attempts at suspending these water-soluble antioxidants in oil with AAFCO (Association of American Feed Control Officials) approved ingredients at concentrations needed for use in antioxidant formulations have been unsuccessful.

### SUMMARY OF THE INVENTION

Recently, lipid soluble catechins (LSC) were identified to have antioxidant properties and maintain solubility in hydrophobic media, including vegetable oils. Oxidative Stability Index (OSI) results of LSC in animal and vegetable fat were promising and, as a result, the material was used in this sequence of trials. These materials, in conjunction with rosemary extract, natural surfactants (lecithin) and chelators (citric acid), were paired in formulas with the goal of creating a more versatile and equal, if not improved, formula with better efficacy.

New dry antioxidant formulations were developed to include not only fat soluble antioxidants, chelators, surfactants, but water-soluble antioxidant extracts from spearmint and rosemary, which provided for an improved level of efficacy, especially at accelerated temperature storage conditions. As part of the antioxidant dry formulations, the chelators were varied and included organic acids, inorganic phosphate, milk whey protein and polyphosphate chelators with a targeted range in pH from 1.5 to 12. Surfactants for the dry formulations included lecithin, but also may include other ionic or non-ionic surfactants that are naturally derived or from non-natural sources, to either solubilize, act as a synergist and enhancing the antioxidant properties, or to further distribute the antioxidant into the desired host matrix.

For all formulations mixed tocopherols includes a single iso-form or a mixture of all structural isomers (alpha, beta, gamma, etc.) of tocopherols and/or tocotrienols.

### BRIEF DESCRIPTION OF THE DRAWINGS

FIG. 1 is a chart of peroxide values (meq/kg diet) of kibbles treated with 1000 ppm of 8 different liquid antioxidant formulations stored at 65° C.

FIG. 2 is a comparison between OSI and PV (time to rancidity) results in sunflower oil treated with antioxidants

### DESCRIPTION OF THE INVENTION

The best antioxidant choice for stabilization of any given product depends on multiple complex factors. One of the factors that affects the selection is the polarity of the antioxidant. The polarity of the antioxidant affects where the antioxidant is located in the product and whether it can interact with free radicals. For example, polar antioxidants are effective in bulk oil situations due to what is referred to as the polar paradox. The polar paradox states that polar antioxidants do not like a non-polar oil matrix and will concentrate at polar interfaces. In a bulk oil these interfaces include air/oil interfaces and water/oil interfaces often in the form of emulsified micelles. A non-polar antioxidant is not as effective as the antioxidant is simply diluted and dispersed in the general oil matrix and not concentrated at the interface. The opposite has been observed with animal protein meals and pet food diets, where non-polar antioxidants have been observed to perform the best. Numerous trials have shown that the polar antioxidants do not perform as well in these meal and diet matrices.

Previous work has attempted to utilize the top antioxidants that performed well in the AOCS Official Method Cd 12b-92 oil stability index (OSI) to identify the best antioxidants for stabilization. As expected in an OSI test which measures stability on bulk oils, the polar antioxidants perform the best due to the polar paradox described earlier. It is common practice in the industry to use the OSI as an antioxidant screening tool. It has been our observations that the OSI is not an appropriate tool for predicting the best antioxidants for meals or diets, and goes counter to accepted practice. Top performing polar antioxidants include examples such as water-soluble green tea, gallic acid, ascorbic acid, etc. In particular, water soluble green tea extracts contain a substantial quantity of the unmodified or natural leaf polyphenols many of which are synthesized into the natural form of catechins (see, e.g., US 2007/0286932). These antioxidants perform very poorly when they are used for meal and diet stabilization. While the best antioxidants for meals and diets are non-polar, there are a limited number of natural non-polar or oil soluble antioxidants. Examples include tocopherols, tocotrienols, camosic acid, etc. There are other non-polar antioxidants but they do not have favorable commercial pricing. The present invention discloses for the first time the use of lipid soluble catechins which have the advantage of being oil soluble, economically viable and suitable for replacing a large amount of conventional antioxidants while still providing effective stabilization of meals and diets. The lipid soluble catechins are shown to provide meal and diet stabilization beyond what is achievable in water soluble forms of catechins.

In the present invention, the antioxidants, individually or in combination, can be added to

the overall diet or to the oil used in the diet.

Tocopherols are traditionally applied to diets in amounts between 50 and 250 ppm. Tocopherols are known to be prooxidants in oils above about 5000 ppm. The ranges of tocopherols applied to oils and diets in the present invention are between 10 ppm and about 250 ppm with a preferred range or between 40 ppm and 240 ppm.

In the present invention, rosemary extracts are used in the range of between 0 and 100 ppm to the diet, with a preferred range of between 0 ppm and 60 ppm to the diet, and between 0 ppm and 360 ppm to the oil with a preferred range of between 0 ppm and 200 ppm to the oil.

In the present invention, lipid soluble catechins are used in the range of between 0 and 120 ppm to the diet, with a preferred range of between 10 ppm and 60 ppm to the diet, and between 10 ppm and 150 ppm to the oil with a preferred range of between 10 ppm and 75 ppm to the oil.

### Example 1—Addition of Lipid Soluble Tea Extract

### Materials and Methods

Liquid antioxidant formulas, the compositions of which are listed Table 1, were applied to extruded kibble in enrobing fat.

TABLE 1
Active ingredients of liquid antioxidant formulations.

| Treatment | Tocopherols | Rosemary | LSC |
|---|---|---|---|
| Name | (%) | (%) | (%) |
| LA 1 | 0 | 0 | 0 |
| LA2 | 24 | 0.1 | 0 |
| LA3 | 20 | 0.1 | 2 |
| LA4 | 17 | 0.1 | 5 |

The chicken fat was treated with 3000 ppm of the liquid antioxidant formulas prior application to the kibble at 4.5%. Palatant was also applied to the kibble at 1%. Finished kibble was stored at 47° C. in individual plastic bags and analyzed for peroxide values (PV) using the FOX II Method (Giilgiin Yildiz, Randy L. Wehling and Susan L. Cuppett. Comparison of four analytical methods for the determination of peroxide value in oxidized soybean oils. Journal of the American Oil Chemists' Society Volume 80, Number 2, 2003, 103-107; Nourooz-Zadeh, Jaffar; Tahaddine-Sarmadi, Javad; Birlouez-Aragon, Ines; and Wolff, Simon P. Measurement of Hydroperoxides in Edible Oils Using the Ferrous Oxidation in Xylenol Orange Assay. J. Agric Food Chem, Bol 43. No. 1. 1995, 17-21) every 2 weeks. Formation of hexanal and 2,4-decadienal was measured at week 4 by gas chromatography (Frankel, EN. Methods to determine extent of oxidation. In: Lipid Oxidation. The Oily Press: Dundee, Scotland. Copyright 1998). The results are presented in Table 2.

TABLE 2
Peroxide values and aldehydes (sum of hexanal and 2,4-
decadienal) levels of the kibble stored at 47° C. for 4 weeks.

| | PV | |
|---|---|---|
| Treatment | (mEq/kg | Aldehydes |
| Name | sample) | (ppm) |
| LA 1 | 4.2 | 156 |
| LA2 | 2.1 | 69 |
| LA3 | 1.4 | 48 |
| LA4 | 1.3 | 43 |

After 4 weeks of storing the kibble at 47° C. the study was terminated as the peroxide values for all of the treatments reached 1 mEq/kg, which is considered an indication of rancidity. As expected, lack of antioxidants (LA1) resulted in highest peroxide value as well as level of aldehydes. More importantly, formulas containing LSC outperformed tocopherol-based antioxidant, as apparent from the peroxides values (1.4, 1.3 for LA3, LA4 vs. 2.1 for LA2) and aldehydes content (48, 43 for LA3, LA4 vs. 69 for LA2).

### Example 2—Addition of Spearmint Extract

Dry antioxidant formulations, listed in Table 3, were added to kibble dry mix with a ribbon blender and extruded in sequence. Water soluble green tea extract (WSGT) standardized to 45% epigallocatechin gallate and 45% other catechins was obtained from Kemin Industries, Inc. (Des Moines, Iowa).

TABLE 3
Active ingredients of dry antioxidant formulations.

| Treatment | Tocopherols | Rosemary | WSGT | LSC | Spearmint |
|---|---|---|---|---|---|
| Name | (%) | (%) | (%) | (%) | (%) |
| DA 1 | 0 | 0 | 0 | 0 | 0 |
| DA2 | 22 | 0.1 | 0 | 0 | 0 |
| DA3 | 11 | 5 | 6 | 0 | 0 |
| DA4 | 11 | 5 | 0 | 6 | 0 |
| DAS | 11 | 5 | 0 | 0 | 5 |

The kibbles were coated with untreated chicken fat at 4.5% and palatant at 1%, and placed in storage at 25° C., 37° C. and 47° C. Samples were analyzed for peroxide values (PV) using the FOX II Method and secondary lipid oxidation products (hexanal and 2,4 decadienal) by gas chromatography. Results are shown in Table 4.

TABLE 4
Peroxide values and aldehydes (sum of hexanal and 2,4-decadienal)
levels of the kibble stored at ambient temperature, 37° C. and 47° C.

| | PV | Aldehydes |
|---|---|---|
| Treatment Name | (mEq/kg sample) | (ppm) |
| | Ambient | |
| | (16 weeks) | |
| DA 1 | 1.9 | 57 |
| DA2 | 1.0 | 28 |
| DA3 | 1.6 | 44 |
| DA4 | 0.7 | 21 |
| DAS | 0.7 | 18 |
| | 37° C. | |
| | (12 weeks) | |
| DA 1 | 7.0 | 296 |
| DA2 | 5.6 | 186 |
| DA3 | 5.8 | 234 |
| DA4 | 2.8 | 77 |
| DAS | 1.5 | 38 |
| | 47° C. | |
| | ( 4weeks) | |
| DA 1 | 4.2 | 156 |
| DA2 | 6.3 | 184 |
| DA3 | 5.3 | 187 |
| DA4 | 1.1 | 29 |
| DAS | 1.1 | 26 |

The inclusion of dry antioxidant into the kibble results in higher oxidative stability as evident from lower peroxide values and aldehyde levels under the storage conditions. Antioxidant formulas containing LSC and spearmint extract performed substantially better than the tocopherol-based formula, especially at higher temperatures. Interestingly, the LSC and WSGT containing formulations demonstrated vast differences in performance, showing that the water-soluble green tea extract did not control oxidation in the pet food matrix tested.

### Example 3—Evaluation of Antioxidant Activity of Lipid Soluble Tea Catechins (LSC) by OSI

The effectiveness of LSC extract in combination with tocopherols, rosemary extract, and lecithin was tested using an animal fat as a matrix. Formulations listed in Table 5 were applied to the fat at 1000 ppm and 3000 ppm levels.

TABLE 5
Composition of formulas

| Treatment | Tocopherols | Rosemary | LSC |
|---|---|---|---|
| Name | (%) | (%) | (%) |
| 0% LSC | 22 | 0.1 | 0 |
| 1% LSC | 21 | 0.1 | 1 |
| 2% LSC | 20 | 0.1 | 2 |
| 3% LSC | 19 | 0.1 | 3 |
| 5% LSC | 17 | 0.1 | 5 |

The induction period of the fat treated with antioxidant formulations (Table 6) was compared to the untreated fat.

TABLE 6
OSI results for chicken fat treated with antioxidant formulas.

| | OSI (hr) | |
|---|---|---|
| Treatment Name | 1000 ppm | 3000 ppm |
| Untreated | 5.9 | |
| 0% LSC | 21.6 | 31.1 |
| 1% LSC | 22.1 | 31.9 |
| 2% LSC | 23.7 | 35.8 |
| 3% LSC | 24.1 | 37.7 |
| 5% LSC | 25.4 | 42.9 |

OSI results show that the antioxidant activity of the formulas increased with higher LSC content. Samples containing 5% LSC applied to the fat at 3000 ppm had the highest induction period among tested formulations.

### Example 4—Evaluation of Antioxidant Efficacy at High Temperatures

Fat samples were treated with 1000 and 3000 ppm of experimental antioxidant formulas having varying ratios of tocopherols, rosemary extract, lipid soluble tea catechins (LSC) and lecithin, as shown in Table 7, and tested in duplicate in the OSI at 100 QC (Table 8).

TABLE 7
LA Prototypes tested in the LSC storage study.

| Treatment | Tocopherols | Rosemary | LSC | Lecithin |
|---|---|---|---|---|
| Name | (%) | (%) | (%) | (%) |
| LSC-1 | 0 | 0 | 0 | 0 |
| LSC-2 | 24 | 0.1 | 0 | 0 |
| LSC-3 | 12 | 6 | 3 | 2 |
| LSC-4 | 18 | 0 | 4 | 2 |
| LSC-5 | 15 | 0 | 7 | 2 |
| LSC-6 | 12 | 0 | 10 | 2 |
| LSC-7 | 0 | 12 | 12 | 2 |
| LSC-8 | 0 | 5 | 18 | 2 |

TABLE 8
OSI results of LSC
formulas in chicken fat.

| | OSI (h) | |
|---|---|---|
| Treatment | 1000 | 3000 |
| Name | ppm | ppm |
| LSC-1 | 6.7 | 6.7 |
| LSC-2 | 30.7 | 56.7 |
| LSC-3 | 35.7 | 62.5 |
| LSC-4 | 35.7 | 54.0 |
| LSC-5 | 39.1 | 57.1 |
| LSC-6 | 16.4 | 34.4 |
| LSC-7 | 14.0 | 30.6 |
| LSC-8 | 34.6 | 50.7 |

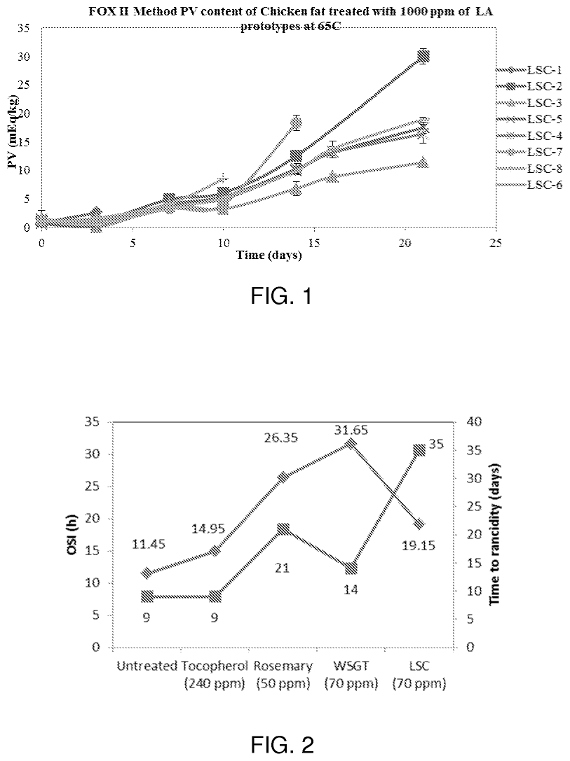

Additionally, 9 g treated poultry fat was weighed into an OSI tube, stored in an OSI unit at 65° C. and connected to air flow tubing. The progress of oxidation was measured by analyzing the rise in peroxide values over time (FIG. 1).

The performance of the liquid formulations containing LSC was equivalent or improved when tested in the OSI at 65° C. Formula LSC-3 out-performed all other formulas at 65° C., and was statistically equivalent in the OSI to the current Naturox® Premium Liquid.

### Example 5—Synergy Between Antioxidants

Experiments were conducted to study the effect of combination of antioxidants on the time to rancidity of sunflower oil. Sunflower oil was treated with tocopherol at 1200 ppm alone and combined with WSGT (350 ppm), rosemary extract (250 ppm) and LSC (350 ppm) and placed in an incubator at 40 QC. Samples of the sunflower oil were periodically analyzed for peroxide values (PV) using the FOX II Method. Time to rancidity (PV?:10 meq/kg oil) was determined for all of the treatments. Results show the increase in stability of sunflower oil treated with combinations of antioxidants (Table 9) in contrast to the treatment with tocopherols alone.

TABLE 9
Synergistic Effect of Antioxidant
Combinations on Time to Rancidity

| | Time to | |
|---|---|---|
| | rancidity | Stability |
| | (days) | increase |
| Tocopherol (1200 ppm) | 9 | |
| Tocopherol (1200 ppm) + WSGT (350 ppm) | 14 | 56% |
| Tocopherol (1200 ppm) + Rosemary (250 | 21 | 133% |
| ppm) | | |
| Tocopherol (1200 ppm) + LSC (350 ppm) | 28 | 211% |

Tocopherols are known to be especially effective in stabilizing sunflower oil. However, a marked and unexpected increase in stability was observed with the addition of lipid soluble catechins. This increase is significantly longer than what was observed with the water-soluble green tea (WSGT) and is counter to what was observed by the OSI results.

### Example 6—Comparison of Stability of Sunflower Oil in OSI and PV Score

According to the American Oil Chemist Society, the Oil Stability Index (OSI) is the point of maximum change in an oil of fat's oxidation under standard conditions. Accordingly, the OSI determines the relative resistance of an oil or fat to oxidation and is an indicator of the length of shelf life for that fat or oil. Experiments were done to evaluate the effect of lipid soluble catechins on the OSI of sunflower oil and on the shelf life of sunflower oil.

Sunflower oil was treated with four different antioxidants: tocopherol at 1200 ppm (total tocopherol concentration); rosemary at 250 ppm (Rosan™ SF 35 from Kemin Industries, Inc., a rosemary extract standardized to 10% carnosic acid); water soluble green tea extract at 35 ppm (standardized to 45% EGCG and 45% other catechins); lipid soluble catechins at 35 ppm (standardized to 74% catechins). Untreated sunflower oil was used at the control. A shelf life study of the same samples at ambient temperature was also conducted. Shelf life time to rancidity was defined as the number of days before the peroxide value (PV) exceeded 10 meq/kg. The results are set out in Table 10.

TABLE 10
Time to Rancidity of Sunflower Oil

| | OSI | Time to rancidity |
|---|---|---|
| Name | (h) | (days) |
| Untreated | 11.45 | 9 |
| Tocopherol (240 ppm) | 14.95 | 9 |
| Rosemary (50 ppm) | 26.35 | 21 |
| WSGT (70 ppm) | 31.65 | 14 |
| LSC (70 ppm) | 19.15 | 35 |

The results show that, surprisingly, the OSI results were not predictive of shelf life for lipid soluble catechins (FIG. 2). The lipid soluble catechins are much more effective at extending shelf life than was expected from the OSI results.

### Example 7—Synergy Between Antioxidants

Experiments were conducted to study the effect of antioxidants alone and in combinations on the peroxide value and 2,4-decadienal values of kibble after 6 weeks at 37° C. In a first set of experiments, the poultry fat used to coat the kibble was either left untreated or treated with 240 ppm tocopherol, 50 ppm rosemary, 70 ppm WSGT, or 70 ppm LSC. The results are shown in Table 11. In a second set of experiments, the fat used to coat the kibble was either left untreated or treated with 240 ppm tocopherol plus 50 ppm rosemary extract, 240 ppm tocopherol plus 70 ppm WSGT, 240 ppm tocopherol plus 70 ppm LSC, 50 ppm rosemary plus 70 ppm WSGT, and 50 ppm rosemary plus 70 ppm LSC. The results are shown in Table 12.

TABLE 11
Effect of Antioxidant Combinations on
Peroxide and 2,4-Decadienal Values

| | PV | 2,4-Decadienal |
|---|---|---|
| Treatment Name | (mEq/kg sample) | (mEq/kg sample) |
| Untreated fat | 19.2 | 22 |
| 240 ppmtocopherol | 14.7 | 18 |
| 50 ppm rosemary | 17.7 | 20 |
| 70 ppm WSGT | 16.7 | 18 |
| 70 ppm WSGT base | 17.7 | 20 |
| 70 ppm LSC | 21.6 | 24 |

TABLE 12
Effect of Antioxidant Combinations on
Peroxide and 2,4-Decadienal Values

| | PV | 2,4-Decadienal |
|---|---|---|
| | (mEq/kg | (mEq/kg |
| Treatment Name | sample) | sample) |
| Untreated fat | 19.2 | 22 |
| 240 ppm tocopherol + 50 ppm rosemary | 14.7 | 17 |
| 240 ppm tocopherol + 70 ppm WSGT | 14.9 | 17 |
| 240 ppm tocopherol + 70 ppm WSGT base | 15.7 | 19 |
| 240 ppm tocopherol + 70 ppm LSC | 15.0 | 17 |
| 50 ppm tocopherol + 50 ppm WSGT | 20.8 | 24 |
| 50 ppm tocopherol + 50 ppm WSGT base | 17.1 | 19 |
| 50 ppm tocopherol + 70 ppm LSC | 16.0 | 18 |

From Table 11 it is seen that the lipid soluble catechins when used alone did not perform as well as the other antioxidants and indeed did not perform as well as leaving the fat untreated. WSGT was one of the better performing antioxidants, which again matched with the observations from the OSI testing. However, when used in combination with tocopherol (Tables 9 and 12), the lipid soluble catechins provided a synergistic protective effect, enabling a reduction in tocopherol to approximately one-fifth of the prior inclusion level without significantly increasing either the peroxide or 1,4-decadienal values. A key goal of the pet food industry has been to reduce the use of tocopherols in the formulations. It has previously been difficult to reduce tocopherol concentrations due to difficulty finding synergistic antioxidants that are effective in a combination that matches the stabilization capability of tocopherols on products under real world storage conditions. In this work we've been able to reduce tocopherol levels up to 80% and still provide similar or better shelf life on a finished pet food diet. This work has shown synergism between tocopherols and LSC in combination or in addition to other antioxidants.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

1. A method for enhancing the stability of a fat-coated pet food composition comprising: adding an antioxidant formulation comprising tocopherol, rosemary, and lipid soluble tea catechins (LSC) to animal fat to form an animal fat coating composition and; coating a pet food composition with the animal fat coating composition.

2. A method according to claim 1 whereby the tocopherol is added to the animal fat in an amount of between 10 ppm to about 250 ppm.

3. A method according to claim 2 whereby the tocopherol is added to the animal fat in an amount of between 40 ppm and 240 ppm.

4. A method according to claim 1 whereby the antioxidant formulation is liquid and the tocopherol is added to the animal fat in an amount of 17-24% by weight of the liquid antioxidant formulation.

5. A method according to claim 1 whereby the antioxidant formula is liquid and is applied to the pet food composition in an amount of 4.5% by weight.

6. A method according to claim 1 whereby the rosemary is added to the animal fat in an amount of up to 100 ppm.

7. A method according to claim 1 whereby the antioxidant formula is liquid and the rosemary is added to the animal fat in an amount of 0.1% by weight of the liquid antioxidant formulation.

8. A method according to claim 1 whereby the antioxidant formula is dry and the tocopherol is added to the animal fat in an amount of 11-22% by weight of the dry antioxidant formulation.

9. A method according to claim 1 whereby the antioxidant formula is dry and the rosemary is added to the animal fat in an amount of 0.1-5% by weight of the dry antioxidant formulation.

10. A method according to claim 1 whereby the antioxidant formula is liquid and the LSC is added to the animal fat in an amount of 2-5% by weight of the liquid antioxidant formulation.

11. A method according to claim 1 further including the step of applying a palatant to the pet food composition.

12. A method for protecting animal fat in pet food from oxidation during rendering, comprising adding an antioxidant composition comprising tocopherol, a rosmarinic acid-containing extract of a Lamiaceae spp. plant, and lipid soluble tea catechins to the fat prior to the rendering process or during the rendering process, whereby the tocopherol is added to the fat in an amount of between 10 and about 250 ppm and the lipid soluble tea catechins are added to the fat in an amount of between 10 and 150 ppm.

13. A method according to claim 12, whereby the plant is selected from the group consisting of basil, mint, rosemary, sage, savory, marjoram, oregano, thyme and lavender.

14. A method according to claim 12 whereby the tocopherol is added to the fat in an amount of between 40 and 240 ppm and the lipid soluble catechins are added to the fat in an amount of between 10 and 70 ppm.

15. An improved fat-coated pet food composition with enhanced stability of the fat coating comprising a tocopherol, a rosmarinic acid-containing extract of a Lamiaceae spp. lipid soluble tea catechins, and a fat-coated pet food, whereby the fat-coating of the pet food comprises between 10 and about 250 ppm of the tocopherol and between 10 and 150 ppm of the lipid soluble tea catechins.

16. An improved fat-coated pet food composition according to claim 15 whereby the fat-coating comprises between 40 and 240 ppm of the tocopherol and between 10 and 75 ppm of the lipid soluble tea catechins.

17. An improved fat-coated pet food composition according to claim 15 which extends the shelf life of the fat-coating in comparison to untreated fat-coating.

18. An improved fat-coated pet food composition according to claim 15 further including a palatability enhancer.

19. An improved fat-coated pet food composition according to claim 15 whereby the extract of a Lamiaceae spp. plant is selected from the group consisting of basil, mint, rosemary, sage, savory, marjoram, oregano, thyme and lavender.

20. An improved fat-coated pet food composition according to claim 15 whereby the fat-coating of the pet food comprises at least 50 ppm of the rosmarinic acid-containing extract.
